(19) United States
(12) Patent Application Publication
(10) Pub. No.: US 2022/0319636 A1
(43) Pub. Date: Oct. 6, 2022

(54) VARIATION POLYGENIC INDEX/SCORE

(71) Applicant: The Trustees of Princeton University, Princeton, NJ (US)

(72) Inventor: Dalton Conley, New York, NY (US)

(73) Assignee: The Trustees of Princeton University, Princeton, NJ (US)

(21) Appl. No.: 17/696,069

(22) Filed: Mar. 16, 2022

Related U.S. Application Data
(60) Provisional application No. 63/166,048, filed on Mar. 25, 2021.

(51) Int. Cl. G16B 20/40; G16B 20/20; G16B 40/00; G16B 50/00; G16B 30/00

(52) U.S. Cl. CPC G16B 20/40; G16B 20/20; G16B 40/00; G16B 50/00; G16B 30/00

(57) ABSTRACT

Disclosed is a method for calculating genetic score based on genotypic information that predicts plasticity in a phenotype. More particularly, disclosed is an algorithm to calculate a particular type of genetic score based on basic genotypic information that is provided by commercially available technologies ranging from “SNP-chips” to whole genome sequencing. Polygenic scores— attempts to summarize the genetic propensity for or risk of a given phenotype (i.e., disease or trait)—have been around for more than a decade. They aim to predict the level of a trait—i.e., how tall or short someone may be or what their blood pressure or BMI might be. The Variation Polygenic Score (“vPGS”) disclosed herein is different. Its purpose is not to predict whether someone who scores higher or lower on the vPGS will be, for instance, heavier or lighter or have a higher or lower IQ. Rather, it is formulated to predict variation. The disclosed vPGS does not predict the mean level but rather the dispersion around that mean. It likely also predicts individual changes in a phenotype over the lifecourse (e.g., whether an individual tends to fluctuate greatly in weight). The disclosed approach is very suited for gene-environment interaction studies: that is, it is a good measure of the genetic propensity to be influenced by the environment for or intervention on a particular trait, disease or other phenotype.

### CROSS-REFERENCE TO RELATED APPLICATIONS

This application claims priority to U.S. Provisional Patent Application 63/166,408, filed Mar. 25, 2021, the entirety of which is incorporated by reference herein.

### BACKGROUND

A wide range of research has shown how outcomes of interest to scientists—e.g., fertility, educational attainment, diseases with marked disparities such as obesity, etc.—are influenced by both an individual's genetic makeup and his or her social environment. This research is broadly referred to as gene×environment (G×E) research.

It has become apparent that most outcomes of interest to social, behavioral and medical scientists, including, e.g., educational attainment, body mass index (BMI), blood pressure, cardiovascular disease, depression, etc., are “polygenic,” i.e., the result of small contributions of many variants across the genome, rather than “monogenic. Polygenic scores or indices—attempts to summarize the genetic propensity for or risk of a given phenotype (e.g., disease or trait)—have been around for more than a decade. They aim to predict the level of a trait, e.g., how tall or short someone may be or what their blood pressure or BMI might be.

The proliferation of polygenic scores as the workhorse tool for studying genetic moderation has run into a problem. Efforts have focused on outcome moderation. That is, the environment's impact on some outcome depending on that individual's genetic propensity to attain that same outcome. Such an environmental impact to be assessed for genetic moderation might be a naturally occurring variation in the physical or social environment. It might be a programmatic or policy intervention, or it might be a medical or pharmaceutic treatment. For instance, pre-K having a larger effect on academic outcomes among children with an already-high genetic propensity towards high educational achievement. These polygenic scores (also referred to as mean PGS, mPGS, or levels PGS) only reflect the influence of genetics on levels of an outcome.

However, no tools exist for describing the environment's impact on some outcome depends on that individual's propensity towards variability in an outcome.

### BRIEF SUMMARY

Disclosed is a method and system for constructing genome-wide summary measures, useful for, e.g., G×E research, that describe the environment's impact on some outcome depends on that individual's propensity towards variability in an outcome, which may be referred to herein as plasticity.

Embodiments of a disclosed method for calculating variation in a genetic score are described herein.

The method includes generating a mean polygenic score (mPGS) by running a regression using phenotypic information to predict an inverse normal transformation for each outcome of one or more outcomes, such that the weights reflect the contribution of each genetic locus of a plurality of genetic loci to the mean level of the outcome, and summing the weights of the regression into the mPGS.

The method also includes generating at least one variance polygenic score (vPGS) by one or more of the following options:
performing a regression to predict a squared Z-score for each outcome of the phenotypic information, and summing the weights of the regressions into a single index for scoring individuals' DNA variants into the variation polygenic score.
performing a regression to predict a statistical measure of spread within a familial relationship of the phenotypic information, e.g., differences in a phenotype among full siblings, and summing the weights of the regressions into a single index for scoring individuals' DNA variants into the variation polygenic score.
performing a mean-variance vQTL analysis for variance heterogeneity of the phenotypic information, and summing the weights of the regressions into a single index for scoring individuals' DNA variants into the variation polygenic score.
performing a mean-variance vQTL analysis for variance effects of phenotypic information, and summing the weights of the regressions into a single index for scoring individuals' DNA variants into the variation polygenic score.

In some embodiments, the generation of vPGS may comprise calculating a statistical measure of spread within a familial relationship of phenotypic information, where the statistical measure of spread comprises standard deviation, Levene's distance, and/or range. In some embodiments, the phenotypic information may include, e.g., height, body mass index, educational attainment, and/or number of children born.

Embodiments of a disclosed system may include a processor and a non-transitory computer readable storage medium. The non-transitory computer readable storage medium contains instructions that, when executed, cause the processor to generate at least to scores, a mean polygenic score (mPGS) and at least one variance polygenic score (vPGS). The mean polygenic score (mPGS) may be generated by running a regression using phenotypic information to predict an inverse normal transformation for each outcome of one or more outcomes, such that the weights reflect the contribution of each genetic locus of a plurality of genetic loci to the mean level of the outcome, and summing the weights of the regression into the mPGS.

The at least one variance polygenic score (vPGS) may be generated via one or more of the following options:
performing a regression to predict a squared Z-score for each outcome of the phenotypic information, and summing the weights of the regressions into a single index for scoring individuals' DNA variants into the variation polygenic score.
performing a regression to predict a statistical measure of spread within a familial relationship of the phenotypic information, and summing the weights of the regressions into a single index for scoring individuals' DNA variants into the variation polygenic score.
performing a mean-variance vQTL analysis for variance heterogeneity of the phenotypic information, and summing the weights of the regressions into a single index for scoring individuals' DNA variants into the variation polygenic score.
performing a mean-variance vQTL analysis for variance effects of phenotypic information, and summing the weights of the regressions into a single index for scoring individuals' DNA variants into the variation polygenic score.

In some embodiments, the statistical measure of spread comprises standard deviation, Levene's distance, and/or range. In some embodiments, the phenotypic information may include height, body mass index, systolic and diastolic blood pressure, number of alcoholic drinks consumed per week, number of cigarettes consumed per day, depression symptomology score, cognitive test score, educational attainment, and/or number of children born.

### BRIEF DESCRIPTION OF THE DRAWINGS

FIG. 1 is a flowchart of an embodiment of a method.

FIG. 2 is a schematic illustration of an embodiment of a system configured to use the disclosed method.

FIG. 3A is a table illustrating the job loss on BMI, with an without mPGS and vPGS interactions.

FIG. 3B is a graph depicting 95% confidence intervals of predicted BMI for treatment and control groups from empirical models for mPGS and vPGS interactions.

FIG. 3C is a set of graphs illustrating coefficient estimates for an event time study (ETS) model for a full sample of respondents (left panel), coefficient estimates for a regression using only respondents in the bottom 50% of vPGS scores (middle panel), and coefficient estimates for a regression using only respondents in the top 50% of vPGS scores (right panel).

FIG. 4 is a table illustrating the impact of education reform on health outcomes.

FIG. 5A is a graph showing 95% confidence intervals for interactions between mPGS and Instrumented Educ16 on Body Size outcomes.

FIG. 5B is a graph showing 95% confidence intervals for interactions between mPGS and Instrumented Educ16 on the Above Threshold version of Body Size outcomes.

### DETAILED DESCRIPTION

Disclosed herein are system and methods for calculating variation in a genetic score based on genotypic information.

The Variation Polygenic Score (“vPGS”) disclosed herein is different from traditional polygenic scores (PGS). Its purpose is not to predict whether someone who scores higher or lower on the vPGS will be, for instance, heavier or lighter or have a higher or lower IQ. Rather, it is formulated to predict variation. That is, if one divided a group of people into two groups, one group having a high vPGS and one group having a low vPGS, the two groups may each have a same average height of five-foot-nine-inches. But those in the low vPGS group may be all clustered around that mean level of 5′9″ (say, +/−2 inches). However, those in the high vPGS group may have a much broader range, say 5′9″+/−4 inches. One might express this in standard deviations, but inches are used for illustrative purposes. Moreover, a traditional PGS for body mass index (BMI) might predict one group to have a BMI of 28 and another group to have one of 23. The disclosed vPGS again does not predict the mean level but rather the dispersion around that mean. It likely also predicts individual changes in BMI over the lifecourse (i.e., whether an individual tends to fluctuate greatly in weight).

The disclosed systems and methods have great utility to drug companies and other experimental settings. Since efficacy of pharmaceuticals (or any treatment) is based on the difference between the treatment group (who receives the drug) and the control group (who receives a placebo), when there is a high “placebo” effect, that makes it all the more difficult to demonstrate a true effect of the treatment. Therefore, companies who run trials try to determine who is likely to demonstrate a big placebo effect and eliminate them from the subject pool.

The disclosed technique takes a different, complementary approach. By scoring potential subjects for, e.g., an experimental blood pressure medication on their vPGS for blood pressure, companies running trials may with to retain only those subjects with a high vPGS—that is, subjects whose genetic architecture likely makes them more “sensitive” to treatments. In other embodiments, it may be advantageous to retain low vPGS subjects, as it might be easier to detect a statistical change that can be attributed to the treatment. This, in turn, may magnify effects, increase statistically power and lower the costs of trials (by requiring fewer subjects to demonstrate a statistically significant effect). This approach need not be limited to pharmaceutical interventions but can be applied, really, to any randomized controlled trial, and there are other applications (e.g., risk screening in clinical and non-clinical settings) where the vPGS should be helpful as well. That is, in some embodiments, one or more individuals may be selected for participation in a trial based on the vPGS score (e.g., having a vPGS score above or below a threshold, as appropriate). In some embodiments, a risk of a particular outcome occurring for an individual may be determined based on the vPGS score (e.g., having a vPGS score above or below a threshold, as appropriate).

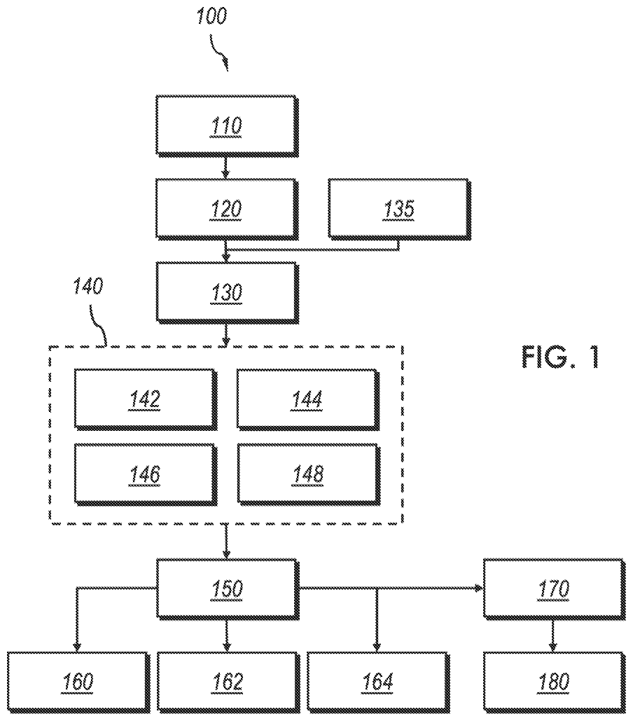

Referring to FIG. 1, disclosed is a method for calculating variation in a genetic score. The method 100 may include determining 110 phenotypic information of one or more individuals, preferably a plurality of individuals, and more preferably 10 or more individuals. This phenotypic information may be collected in a variety of ways. In some embodiments, the phenotypic information may include genotypic information. The genotypic information may be basic genotypic information, such as that provided by commercially available technologies ranging from “SNP-chips” to whole genome sequencing.

The method may include receiving 120 the phenotypic information of one or more individuals. This may be received from, e.g., a database, a remote computer or server, or any other appropriate means for providing the information.

The method may include generating 130 a mean polygenic score (mPGS) by running a regression using phenotypic information to predict an inverse normal transformation for each outcome of one or more outcomes, such that the weights reflect the contribution of each genetic locus of a plurality of genetic loci to the mean level of the outcome, and then summing the weights of the regression to determine the mPGS.

In some embodiments, the method may include selecting or defining 135 the one or more outcomes, where the one or more outcomes are present in the phenotypic information. In FIG. 1, this step is shown as being performed just prior to step 130, for generating a mean polygenic score (mPGS). However, it will be understood that this step can be performed at other times as well.

It will be understood that almost any outcome can be selected or defined, based on the data being collected and the purpose for which the data is being collected. In some embodiments, the selected or defined outcomes may include height, body mass index, systolic and diastolic blood pressure, number of alcoholic drinks consumed per week, number of cigarettes consumed per day, depression symptomology score, cognitive test score, educational attainment, and/or number of children born. In some embodiments, the selected or defined outcome may include an outcome that relates to a degree of susceptibility or reactivity to a chemical compound (e.g., a pharmaceutical drug, a food product, etc.) or a particular disease (e.g., diabetes, heart disease, cancer, etc.).

The method will include generating 140 at least one variance polygenic score (vPGS). This information may be generated in a variety of ways, which may include one or more of the following options.

A first option is to perform 142 a regression to predict a squared Z-score for each outcome of the phenotypic information, and then summing the weights of the regressions to determine a single index (a first type of vPGS) for scoring individuals' DNA variants into the variation polygenic score.

A second option is to perform 144 a regression to predict a statistical measure of spread within a familial relationship of the phenotypic information, for each outcome, and then summing the weights of the regressions to determine a single index (a second type of vPGS) for scoring individuals' DNA variants into the variation polygenic score. In some embodiments, the statistical measure of spread comprises standard deviation, Levene's distance, or range.

A third option is to perform 146 a mean-variance quantitative trait loci (vQTL) analysis for variance heterogeneity of the phenotypic information, for each outcome, and then summing the weights of the regressions to determine a single index (a third type of vPGS) for scoring individuals' DNA variants into the variation polygenic score.

A fourth option is to perform 148 a mean-variance quantitative trait loci (vQTL) analysis for variance effects of phenotypic information, for each outcome, and then summing the weights of the regressions to determine a single index (a fourth type of vPGS) for scoring individuals' DNA variants into the variation polygenic score.

In some embodiments, only a single option (142, 144, 146, 148) is used. in some embodiments, two or more options are used. In some embodiments, all four options are used.

In some embodiments, the at least one vPGS will capture genetic contributions to variability in an outcome, distinct from genetic contributions to levels of an outcome. In some embodiments, the method will include determining 150 if the vPGS captures genetic contributions to variability in an outcome, distinct from genetic contributions to levels of an outcome.

Various tools may be used to explore whether vPGS captures genetic contributions to variability in an outcome, distinct from genetic contributions to levels of an outcome.

First, one can estimate the following linear regression,

Yᵢ=α+β₁PGSᵢ+γXᵢ+εᵢ (1)

where i indexes a respondent, PGS indicates the levels PGS (mPGS) or a variance PGS (vPGS), and Y is levels of the outcome trait (converted to the standard normal scale). X includes the first 5 principal components (PCs). The coefficient of interest is β₁—one would expect the levels PGS to significantly predict levels of a trait.

In turn, single nucleotide polymorphisms (SNPs) are a mix of four types: (1) SNPs that affect neither levels of an outcome nor variance in an outcome, (2) SNPs that affect levels of an outcome but not its variance, (3) SNPs that affect variance in an outcome but not levels of an outcome, and (4) SNPs that affect both variance in and levels of an outcome. To serve as a useful new tool for G×E research, an ideal plasticity score will mostly contain SNPs of type three—those that affect variance without necessarily affecting mean. Therefore, β₁ when one regresses levels of an outcome on a vPGS should ideally be close to zero and not statistically significant.

Second, one can examine whether the patterns of correlation after constructing the vPGS in each sample are also present in the underlying weights that summarize each SNP's contribution. One can use linkage disequilibrium score (LD) score regression (see Bulik-Sullivan et al., “LD score regression distinguishes confounding from polygenicity in genome-wide association studies”, Nature genetics, 47(3):291-295 (2015), incorporated by reference herein in its entirety) for two purposes. First, one can use the technique to compare the heritability of levels of an outcome to the heritability of plasticity in that outcome measured using the four disclosed vPGS techniques disclosed herein. Further, one can compare the underlying genetic correlations between (1) levels and variability for each outcome, (2) across outcomes in levels and variability.

In some examples, one or more of the polygenic scores for plasticity may fail to summarize genetic contributors unique to variability in an outcome, while one or more of the polygenic scores does not fail, and captures more distinctive genetic contributions.

In some embodiments, a first data set is used as a training data set, and a model is trained using the training data set to arrive at one or more polygenic scores determining to capture genetic contributions to variability in an outcome, distinct from genetic contributions to levels of an outcome. That trained model is then used against other data sets.

Thus, in some embodiments, one or more of the polygenic scores (including the weights) determining to capture genetic contributions to variability in an outcome, distinct from genetic contributions to levels of an outcome, are selected 170 for use against a different data set.

In some embodiments, the selected one or more polygenic scores (including the weights) are applied 180 to a different data set.

In some embodiments, the vPGS scores may be displayed 160, e.g., on a display screen, or stored 162, e.g., on non-transitory computer readable medium, or transmitted 164 to a remote processor, such as a mobile device, computer, or server.

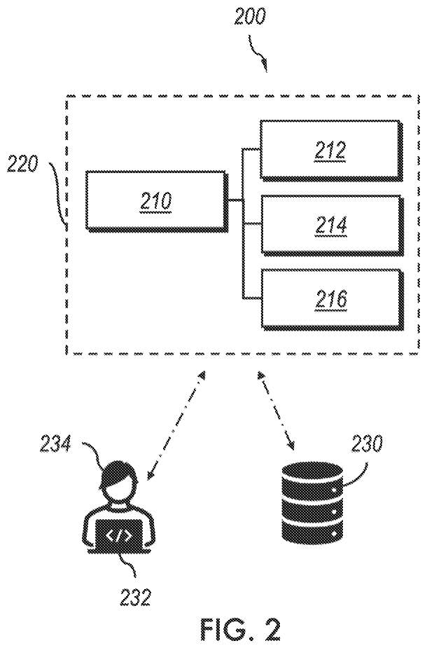

Also disclosed is a system that can be configured to utilize the disclosed method. As seen in FIG. 2, the system 200 may include a processor 210, which may be operably connected to non-transitory computer readable medium 212, a display 214, and/or a communication transceiver 216. The processor 210 will generally be within a device 220. The device 220 may be, e.g., a laptop, a server, a desktop computer, a mobile phone, etc.

The non-transitory computer readable storage medium 212 may contain instructions that, when executed, cause the processor 210 to perform a number of tasks, including performing an embodiment of the method as disclosed herein.

In some embodiments, the processor may be configured to generate a mean polygenic score (mPGS) by running a regression using phenotypic information to predict an inverse normal transformation for each outcome of one or more outcomes, such that the weights reflect the contribution of each genetic locus of a plurality of genetic loci to the mean level of the outcome and summing the weights of the regression into the mPGS.

In some embodiments, the processor may be configured to generate at least one variance polygenic score (vPGS).

In some embodiments, the processor may be configured to perform a regression to predict a squared Z-score for each outcome of the phenotypic information, and then sum the weights of the regressions into a single index for scoring individuals' DNA variants into the variation polygenic score.

In some embodiments, the processor may be configured to perform a regression to predict a statistical measure of spread within a familial relationship of the phenotypic information, and then sum the weights of the regressions into a single index for scoring individuals' DNA variants into the variation polygenic score.

In some embodiments, the processor may be configured to perform a mean-variance vQTL analysis for variance heterogeneity of the phenotypic information, and then sum the weights of the regressions into a single index for scoring individuals' DNA variants into the variation polygenic score.

In some embodiments, the processor may be configured to perform a mean-variance vQTL analysis for variance effects of phenotypic information, and then sum the weights of the regressions into a single index for scoring individuals' DNA variants into the variation polygenic score.

In some embodiments, the phenotypic information used to generate a vPGS may be on a database, e.g., a database 230, a remote computer 232, or any other appropriate means for providing the information.

In some embodiments, the device 220 is configured to receive and/or send data to the database 230, remote computer 232, etc.

In some embodiments, a user 234 may select or define outcomes for use in various aspects of the disclosed method. The user may make such selections or definitions directly on the device 220, or may enter such information remotely, e.g., via remote computer 232.

In some embodiments, the method may include making a determination of risk when the vPGS score is above a vPGS threshold. In some embodiments, the method may include making a determination of risk when the mPGS is above an mPGS threshold and the vPGS score is above a vPGS threshold. In some embodiments, the method may include making a determination of risk when the mPGS score is below an mPGS threshold and the vPGS score is above a vPGS threshold.

In some embodiments, the method may include selecting a participant in a study based on a vPGS score being above a vPGS threshold. In some embodiments, the method may include selecting a participant in a study based on a vPGS score being below a vPGS threshold.

### Example 1 (UK Biobank)

To build the two types of scores for comparison—(1) a typical PGS (mPGS) for levels of an outcome and (2) a vPGS for variability in an outcome—a dataset containing about 500,000 individuals from across the United Kingdom, the UK Biobank, can be used. The sample was limited to respondents who passed quality control and were of British ancestry, using information provided by the UK Biobank, leaving us with 408,219 in our final analytic sample.

This size of the UK Biobank allows one to divide the sample into training and test sets while still maintaining sufficient statistical power for fitting GWAS and vQTL. In this example, training and test sets were produced by randomly sampling respondents. 80% of the British subsample of the UK Biobank was included in the training set; the remaining 20% made up the test set.

Four outcomes were analyzed: height, body mass index (BMI), educational attainment, and number of children ever born, a measure of fertility. The inverse normal transformations of the outcomes were calculated. Traits were also z-scored to create a second set of dependent variables, used in the Squared Z-score analyses. Unless specified as z-scored, a trait/outcome should be assumed to be inverse normal transformed.

For each outcome, first a regression was run predicting the inverse normal of that outcome, such that the weights reflect the contribution of each genetic locus to the mean level of the outcome. These regressions were performed using the software PLINK (version 1.9), controlling for age, sex, their interaction, and the first 40 PCs. Terms with higher order age variables (e.g., age², age²*sex), were excluded due to issues with multicollinearity. These regression weights are referred to as weights for Levels PGS, and they correspond to the traditional tools used in G×E research.

A set of second identical regressions predict the Squared Z-score, rather than inverse normal, of the outcome. Since the z-score is squared, values which are the same number of standard deviations above or below the mean will receive the same value. Thus, the regression predicts distance from the mean, rather than the mean-level itself, though, as we argue above, this will still be correlated with the mean. The weights and polygenic scores produced by these regressions are referred to as Squared Z.

Third, regressions were run for each outcome on the sibling subsample of the UK Biobank, which includes 22,657 white British sibling pairs. For each sibling pair, the intra-sibling mean and SD were calculated. The SD was then residualized with the mean, and this new residualized standard deviation was used as the outcome variable. Since each sibling pair was represented twice in the data, only one member of each sibling pair was used in the final regressions. The weights and polygenic scores produced by this method are referred to as Sibling SD.

Fourth, a Mean-Variance vQTL analysis using Levene's test for variance heterogeneity was run using OmicS-data-based Complex trait Analysis (OSCA) (see cnsgenomics.com/software/osca). The Levene's test does not estimate the effect size and standard error, but rather assesses the equality of variances between sample groups (in this case, those that do and do not have a given allele). Thus, this approach re-scales the test statistics (p value) to effect size and standard error using Z-statistics. The weights and polygenic scores produced by this method may be referred to as Levene's.

Fifth, a Mean-Variance vQTL analysis using heteroskedastic linear mixed models was similarly used. This method produces additive (mean) and variance effects. It also allows one to derive what are termed dispersion effects, which are variance effects that are independent of the mean effect. The weights produced by these dispersion effects may be used in subsequent analyses, and may be referred to as HLMM.

Finally, to ensure that results comparing the different vPGS were due to true differences between the scores, and not due to differences that arise from the smaller sample size and lower precision in the sibling-based method, for every vQTL or GWAS analysis run on the full sample an analogous analysis was run on a randomly-chosen subsample, where the number of respondents was set to be equal to the number of sibling pairs in the UK Biobank.

The analyses show that two of the polygenic scores for plasticity—one constructed using the Squared Z-score of an outcome; the other constructed using Levene's test for variance heterogeneity—fail to summarize genetic contributors unique to variability in an outcome. However, two of the polygenic scores for plasticity—one summarizing “dispersion” effects; the other constructed from sibling variation—capture more distinctive genetic contributions.

### Example 2 (Constructing vPGS in the Health and Retirement Study (HRS))

Using the weights from Example 1, one can construct vPGS in a dataset from the HRS. The FIRS sample is restricted to (1) self-identified European Americans, who (2) pass the FIRS preprocessing procedure and are within 2 standard deviations of the mean of the first two principal components of their racial/ethnic group. This leaves N=10,554 respondents. The replication code contains details on the outcome variable construction; most notably, since the FIRS is time-varying with several waves, we took the most recently observed value of the outcome for each respondent.

The first question that arises when using plasticity scores for G×E research is: is the plasticity score simply capturing genome-wide contributions to levels of an outcome, rather than capturing genome-wide contributions to variability in an outcome? If the plasticity score looks very similar to social scientists' standard tool for G×E research, it is less useful as a new tool for capturing distinctive forms of genetic moderation. Table 1, below, summarizes the results of a model, or whether the plasticity score significantly predicts levels of an outcome.

TABLE 1
(Significance of mPGS and plasticity scores in predicting levels of a trait)

| | Dataset: | | | | | | | |
|---|---|---|---|---|---|---|---|---|
| | HRS | | | | UK Biobank | | | |
| Outcome: | BMI | Educ. | Height | NEB | BMI | Educ. | Height | NEB |
| mPGS | S | S | S | M | S | S | S | S |
| Squared Z vPGS | S | S | S | N | S | S | S | N |
| Levene vPGS | S | S | N | N | S | S | N | N |
| HLMM vPGS | N | S | M | N | N | S | S | S |
| Sibling SD vPGS | N | S | N | N | S | S | N | N |

(S = Significant, M = Marginal, N = Not Significant)

The left hand side shows the results in the smaller sample size HRS; the right panel the results in the larger sample size UK Biobank test set. Each subcolumn represents one of the four outcomes of interest to demographers: height; BMI; education; number of children ever born (NEB). The top row (mPGS) shows that, as expected, the levels PGS predict levels of an outcome (though the relationship with fertility in FIRS is weaker than for height, BMI, and education). Moving downwards, across both samples, the Squared Z plasticity score performs the least well in that the plasticity score significantly predicts levels of an outcome for all outcomes except for number of children ever born. The Sibling SD score and HLMM perform best in the HRS test set.

Overall, the results show that researchers hoping to use plasticity scores for gene-environment research should be careful to choose one of the tools that captures distinctive genetic contributions to plasticity apart from genetic contributions to an outcome's mean.

These results show that when aggregating weights from the different vQTL methods to produce a polygenic score for plasticity, some of the scores—most notably, the Squared Z vPGS and one based on results from Levene's test—perform similarly to an mPGS in predicting levels of a trait. As a result, they are less useful as tools for examining certain forms of gene-environment interplay since they fail to capture distinctive genetic contributions to variability.

Here, LD score regression can be used to examine two features of the genetic architecture of plasticity. First, the univariate heritability of each of the four outcomes can be examined—first, levels of an outcome and second, plasticity in that outcome. Table 2 shows the results. The first column, in line with past findings, shows the highest heritability for height followed by BMI, education, and finally number of children ever born. One can see that plasticity as defined using the Squared Z score and Levene's test have higher heritability, which as seen previously shows could be related to capturing levels-relevant genetic contributions; both the sibling standard deviation method and dispersion effects from HLMM capture plasticity in a way that leads to estimates of zero heritability. Interestingly, focusing on the Squared Z estimates, one can see that the rank order of heritability changes. That is, levels of height are the most heritable, followed by BMI, then education, and fertility is the least heritable. With the Squared Z method, plasticity in BMI was found to be more heritable than plasticity in height.

Table 2 (Heritability of each outcome: levels versus plasticity The cells with zero are heritabilities where, due to sampling variability, the heritability was low enough to be close to zero or negative)

| | Heritability | | | | |
|---|---|---|---|---|---|
| Outcome | Levels | Squared Z | Levene's | Sibling | HLMM |
| Height | 0.40 | 0.0028 | 0.28 | 0.00 | 0.00 |
| BMI | 0.20 | 0.0406 | 0.01 | 0.00 | 0.00 |
| Education | 0.12 | 0.0095 | 0.00 | 0.00 | 0.00 |
| Number Ever | 0.03 | 0.0033 | 0.00 | 0.00 | 0.00 |
| Born | | | | | |

Second, focusing on the Squared Z score that had non-zero heritability across outcomes, one can examine the genetic correlation between (1) levels of each outcome (see Bulik-Sullivan, et al., “An atlas of genetic correlations across human diseases and traits”, Nature genetics, 47(11):1236 (2015), incorporated by reference in its entirety herein, (2) plasticity in each outcome, and (3) levels and plasticity.

Notably, these genetic correlations are prior to estimating the scores in a sample, so reflect a shared genetic architecture between contributors to levels of an outcome and contributors to variability in an outcome.

Table 3 (approximate genetic correlation between the levels PGS (mPGS) and the Squared Z vPGS for each of the outcomes. It shows that the weights for the levels PGS for that trait are significantly correlated with the weights for the Squared Z vPGS)

| | | | | | |
|---|---|---|---|---|---|
| Weights | Height | 0.25 | 0.25 | 0.50 | 1 |
| from | Education | 0.10 | −0.25 | 1 | 0.50 |
| Z-score | NEB | 0.50 | 1 | −0.25 | 0.25 |
| vPGS | BMI | 1 | 0.50 | 0.10 | 0.25 |
| | | BMI | NEB | Education | Height |

Weights for mPGS

The correlation close to 1 on the diagonal shows that there is a high degree of overlap in the weights estimated using each method.

TABLE 4
(Between-trait patterns of genetic correlation for the levels PGS)

| | | | | |
|---|---|---|---|---|
| NEB | 0.25 | −0.33 | −0.10 | 1 |
| Height | −0.25 | 0.25 | 1 | −0.10 |
| Education | −0.50 | 1 | 0.25 | −0.33 |
| BMI | 1 | −0.50 | −0.25 | 0.25 |
| | BMI | Education | Height | NEB |

TABLE 5
(Between-trait patterns of genetic
correlation for the Squared Z vPGS)

| | | | | |
|---|---|---|---|---|
| NEB | 0.50 | −0.25 | 0.10 | 1 |
| Height | 0.10 | 0.50 | 1 | 0.10 |
| Education | −0.10 | 1 | 0.50 | −0.25 |
| BMI | 1 | −0.10 | 0.10 | 0.50 |
| | BMI | Education | Height | NEB |

The analysis investigates whether there are similar patterns of cross-trait genetic correlation in variability in addition to levels. The results show that the patterns are similar except for the relationship between BMI and height. One exception is that levels of height and BMI are negative genetically correlated (in other words, those with genetic propensities to be taller also have genetic propensities towards lower BMI) yet plasticity in height and BMI is positively correlated. Without being held to any particular theory, this relationship could reflect differential sensitivity to environmental inputs to growth.

Taken together, the results show that the Squared Z vPGS and Levene's test-based vPGS are less useful for social scientists looking for a new tool to examine gene-environment interplay. Both significantly predict levels of an outcome across four diverse traits (height; BMI; education; number of children ever born). The Squared Z-score also exhibits patterns of underlying genetic correlation similar to those between levels of a trait. In contrast, the sibling standard deviation method and dispersion weights show better properties in capturing distinctive genetic contributions to plasticity.

### Example 3 (Impact of Late-Career Job Loss and Genotype on BMI)

As discussed herein, to overcome the challenge of lower power to detect individual effects in the context of multiple hypothesis testing, researchers have used whole-genome polygenic scores (PGSs) constructed from well-powered genome-wide association studies (GWAS) that summarize the genomic contribution to a trait or disease across common variants in the genome. That is, PGSs aggregate thousands of genome-wide genetic influences on a phenotype into a single index using results from GWAS that estimate the association between genetic variants and the conditional mean of a phenotype, which we refer to herein as mGWAS. However, PGSs constructed from mGWAS may not capture the impact of loci that contribute to within-individual variance in an outcome that are more responsive to environmental stimuli (i.e., variance quantitative loci or vQTLs). Since estimating genetic contributions to within-person variability is hindered by a lack of large datasets with genotype data and longitudinal phenotypic data on participants, researchers have developed methods that can detect population-level variance effects that are not driven by mean effects, which may sometimes be referred to as vGWAS.

Here, summary statistics from both mGWAS and vGWAS are applied to construct whole genome PGSs for outcomes, such as BMI, that capture mean effects (mPGS) and variance effects (vPGS). Evaluating both measures in a G×E framework is necessary because environmental shifts may moderate individuals' propensity for higher or lower BMI, and/or their propensity towards changes in BMI or BMI plasticity.

The data in this example came from the U.S. Health and Retirement Study (HRS). The focus was specifically on business closures because they are typically the byproduct of external, firm level decisions to restructure or relocate businesses and are therefore considered more exogenous than layoffs or firings, which may be correlated with unobserved health or worker characteristics that could bias G×E estimates. The majority of G×E interaction studies use endogenous measures of the environment that cannot address the non-random distribution of genes across environments. This is important because G×E interactions can, in that case, be proxying a different, unmeasured E that is interacting with G, or G×G interactions (i.e., epistasis) or even E×E (if the measured genes proxy other environments). Specifically, in the case of job loss being endogenous (e.g., for cause), such a measure could be intertwined with a host of unobserved genetic or environmental influences that are associated with health and changes in BMI.

To address this, the disclosed empirical strategy interacts business closures with, respectively, an mPGS and vPGS in a regression-adjusted semiparametric difference-in-differences (DiD) propensity score matching framework that compares the BMI of those before and after an involuntary job loss with a control group that was not laid off. Combining propensity score matching with DiD estimation makes the model more robust to selection on observables and unobservables with time invariant effects (e.g., ability or worker preferences). This is necessary because although business closures are plausibly more exogenous than layoffs or firings, it is still possible that workers with unhealthy behaviors or poor health, for example, could select into more vulnerable or volatile industries.

In the context of older workers in the U.S., one may focus on changes in BMI for, e.g., two reasons. First, BMI is an inexpensive, non-invasive proxy measure of adiposity that is available for all FIRS waves and is predictive of metabolic syndrome and other more difficult to measure anthropomorphic measures like abdominal adiposity that increase risk for cardiovascular disease and type 2 diabetes. In older adults, unintentional weight loss or frailty can also be harmful and indicative of decreased resistance to stressors, resulting in greater vulnerability to disease and disability. Past studies have found slight increases in BMI from unemployment, particularly in middle-aged workers, but there is no consensus, and some studies that look at the causal impact of job loss on BMI or related health outcomes have been unable to locate an average treatment effect.

Second, changes in BMI may be focused on because BMI is currently the most well studied phenotypic trait in vGWAS. Previous meta-analyses of mGWAS have identified more than 100 genome-wide significant loci associated with BMI. The largest cluster of highly significant genetic variants is located in the FTO (fat mass and obesity associated) gene region on chromosome 16. Studies suggest FTO polymorphisms increase obesity risk through subtle changes in food intake and preference and affect pathways in the central nervous system that regulate appetite. In particular, the SNP rs1421085 underlies the association between the FTO locus and obesity via activation of IRX3 and IRX5, which play a role in the differentiation of adipocyte subtypes. Recent vGWAS have found evidence for loci with variance effects on BMI located in genes responsible for adipocyte differentiation (PPARG) and genes implicated in the pathology of obesity, diabetes, atherosclerosis, and cancer (FTO, PPARG, CCNL1, TCF7L2, ZNF668, GIPR). Most BMI-associated loci have their largest impact early in life or during adolescence, although a few loci, which have also been associated with type 2 diabetes or coronary artery disease, exhibit stronger effects in older adults. Past studies have also found genetic moderation of social aspects of the environment that affect BMI, including lifetime socioeconomic status (SES), social norms, birth cohort, and institutional policies.

The HRS is a nationally representative, longitudinal panel study of individuals over the age of 50 and their spouses that is sponsored by the National Institute on Aging (NIA U01AG009740) and conducted by the University of Michigan^{77,78}. Launched in 1992, the HRS introduces a new cohort of participants every six years and interviews around 20,000 participants every two years. To maximize sample size, data was compiled from 13 waves (1992-2016). Information on job loss and smoking behavior was obtained from the 1992-2016 Public Use Core Files; demographic and socioeconomic data came from the RAND Data File (version P).

Genotype data on ˜15,000 HRS participants was collected from a random subset of the 26,000 total participants that were selected to participate in enhanced face-to-face interviews and saliva specimen collection for DNA in 2006, 2008, and 2010. This sample was restricted to individuals of European ancestry who were between the ages of 50-70 who reported working part-time or full-time in the previous wave and who were not self-employed. The final sample consists of 3,938 workers with 11,932 observations.

Genotyping was conducted by the Center for Inherited Disease Research (CIDR) in 2011, 2012, and 2015 (RC2 AG0336495, RC4 AG039029). Genotype data on over 15,000 HRS participants was obtained using the Ilumina HumanOmni2.5 BeadChips (HumanOmni2.5-4v1, HumanOmni2.5-8v1), which measures ˜2.4 million SNPs. Genotyping quality control was performed by the Genetics Coordinating Center at the University of Washington, Seattle, Wash. Individuals with missing call rates >2%, SNPs with call rates <98%, HWE p-value <0.0001, chromosomal anomalies, and first-degree relatives in the FIRS were removed. Imputation to 1000G Phase I v3 (released March 2012) was performed using SHAPEIT2 followed by IMPUTE2. The worldwide reference panel of all 1,092 samples from the Phase I integrated variant set was used. These imputation analyses were performed and documented by the Genetics Coordinating Center at the University of Washington, Seattle, Wash.

Principal component (PC) analysis was performed on a selected set of independent SNPs to identify population group outliers and to provide sample eigenvectors as covariates in the statistical model to adjust for possible population stratification and were provided by the HRS. The European ancestry sample included all respondents that had PC loadings within ±one standard deviations for eigenvectors one and two in the PC analysis of all unrelated study subjects and who self-identified as White on survey data. A second set of principal components was then calculated within the European ancestry sample to further account for any population stratification within the sample. The genotype sample has been defined by the HRS and is available on dbGaP.

Means and matching statistics for covariates by treatment group, control group, and matched control group can readily be determined. After matching, covariates should be balanced with little to no significant differences remaining. In this example, both the standardized bias and two-sample t-tests for equality of the means to check for significant differences between covariates for both groups.

The standardized bias compares the distance between the marginal distributions, or the difference in sample means between the treated (X̅_{T}) and matched control (X̅_{C}) subsamples as a percentage of the square root of the average of the sample variances in both groups for a covariate X:

$${SB}_{x} = 100 \cdot \frac{{\overline{X}}_{T} - {\overline{X}}_{C}}{\sqrt{\frac{1}{2} ( {σ}_{XT}^{2} - {σ}_{XC}^{2} )}}$$

Before matching, individuals affected by a business closure have lower socioeconomic standing, were less likely to have health insurance, and report worse mental health and health behaviors than continuously employed individuals. Labor statistics show they were more likely to work part time, for smaller firms, in the agriculture/fishing/farming, construction/mining, manufacturing, or trade industries, were more likely to be blue collar, and have lower job tenure than workers in the control group. We do not see any significant differences in BMI between treatment and control groups.

After matching, covariates are more balanced overall, and the standardized biases for the majority of variables are at or below 5%, which indicates that mean differences between the treatment and control group are small and the balancing procedure was effective. Notable exceptions include mean differences in education, industry, household income, access to health insurance, and smoking behavior. To minimize any remaining differences between groups, one can control for all covariates in the empirical model. Importantly, significant difference in the mPGS or vPGS between treatment and control groups are not seen before or after matching, indicating the absence of gene-environment correlation (rGE), or evidence of selection into the treatment group by underlying genetic predisposition.

Mean Polygenic Score (mPGS) Construction

A linear mPGS was calculated for the HRS sample based on a GWAS of 457,824 European ancestry individuals in the UK Biobank. Imputed HRS genotype data were accessed through dbGap (phs000428). The mPGS BMI score was constructed in PRSice by taking a weighted sum across the number of SNPs (n) of the number of reference alleles x (zero, one, or two) at that SNP multiplied by the effect size for that SNP (β):

$${PGS}_{i} = {\sum }_{j = 1}^{n} {β}_{j} {x}_{ij} .$$

GWAS summary statistics were pruned for linkage disequilibrium (LD) using the clumping procedure in PLINK (R²=0.1, range=1000 kb). Since these GWAS summary statistics were pre-clumped, no LD-clumping or p-value threshold was implemented in PRSice. After LD clumping was applied, 90,326 SNPs were used to construct the BMI mPGS. The mPGS was standardized to have a mean of zero and a standard deviation of one for all analyses.

Because this approach incorporates mPGS and vPGS into a G×E interaction model, it is important to use a vPGS that captures variance effects that are distinct from mean effects. To decorrelate the mean and variance effects, Young et al, “Identifying loci affecting trait variability and detecting interactions in genome-wide association studies. Nat. Genet. 50, 1608-1614 (2018) (incorporated by reference in its entirety herein) proposed a dispersion effects test that can identify differences in the variance of the GWAS sample as a whole that are not driven by mean effects at the SNP level. One can use the dispersion weights from Young to construct vPGS in the HRS.

The predictive performance of mPGS for BMI in the HRS and other population-based samples has been well studied. To evaluate the predictive performance of the vPGS, a Double Generalized Linear Model (DGLM) can be fitted to allow one to assess the association between the vPGS and the between-individual variance in BMI in a UK Biobank (UKB) test sample that is independent of our HRS testing sample. The dispersion vPGS is significantly associated with the population variance in BMI in the UKB test sample (p=1.01E-04), and this association holds after controlling for the mPGS (p=1.44E-04).

Construction of the Variance Polygenic Score (vPGS)

BMI vPGS was calculated for HRS participants of European ancestry. SNP weights in the vPGS were based on dispersion effects estimated in the UKB using the heteroskedastic linear mixed model (HLMM) approach. Pre-pruned HLMM summary statistics were obtained from prior work. Additional LD-clumping or p-value thresholding to filter variants was not performed. A total of 242,870 SNPs remained in the vPGS model after overlapping the HLMM summary statistics and HRS genotype data. The vPGS was constructed in PRSice and standardized to have a mean of zero and a standard deviation of one for all analyses.

Validation of the Variance Polygenic Score (vPGS)

Performance of the vPGS was assessed using European ancestry UKB samples identified from genetic PCs. To avoid overfitting, HRS samples were not used for model validation. Quality control procedures for the UKB genetic data have been described elsewhere. Participants recommended by UKB (data field 22010), those with conflicting genetically-inferred (data field 22001) and self-reported sex (data field 31), and those who withdrew from the study, were excluded. UKB participants (N=406,873) were randomly apportioned into training (N=325,498) and testing sets (N=81,375), with an 80-20 split. The HLMM approach was applied to estimate the dispersion effect of each SNP on BMI using samples in the training set, controlling for sex, age, age², age³, age×sex, age²×sex, age³×sex, genotyping array, and the first 40 genetic PCs. Related and unrelated samples in the training set were analyzed separately and performed fixed-effect meta-analysis to combine the results. Related samples were inferred from genetic kinship (third-degree relatives or higher; data field 22021). Random effects were included to account for genetic relatedness in the analysis of related samples. We then pruned SNPs and used dispersion effect estimates to generate vPGS for samples in the testing set.

A Double Generalized Linear Model (DGLM) was then fitted to associate the vPGS with the between-individual BMI variance in testing samples. The DGLM takes the form of

BMIᵢ=γ₀+γ₁Gᵢ+XᵢΘ+εᵢ,εᵢ˜N(0,exp(α₀+α₁Gᵢ+Xᵢϕ)),

where BMIᵢ denotes the inverse normal-transformed BMI of individual i, Gᵢ is the vPGS of individual i, Xᵢ is the vector of covariates including sex, age, age², age³, age×sex, age²×sex, age³×sex, genotyping array, and the first 40 genetic principal components. Here, α₁ quantifies the effect of vPGS on the variability of BMI and is the parameter of interest in this analysis. The vPGS was standardized with a mean of 0 and a variance of 1. DGLM was fitted using the dglm package in R.

To assess the performance of vPGS after adjusting for the effect of mPGS, a standard, mean-effect GWAS of BMI was performed on the training set and used the effect estimates to generate mPGS for the testing samples. GWAS summary statistics were pruned for LD using the clumping procedure in PRSice (R2=0.1, range=250 kb) when calculating the mPGS. The same DGLM model as above was then fitted with the mPGS added to the vector of covariates. The mPGS was standardized with a mean of 0 and a variance of 1.

Treatment and Control Groups

For each observation, information from two waves—before and after treatment—was used. Before treatment (t−2), all respondents were working for pay either full- or part-time. At the following FIRS interview two years later (t), respondents in the treatment group report they were no longer working for their previous-wave employer. These respondents were asked why they left their employer. Possible answers included ‘business closed’, ‘laid off/let go’, ‘poor health/disabled’, ‘quit’ ‘family care’, ‘better job’, ‘retired’, ‘family moved’, ‘strike’, ‘divorce/separation’, ‘transportation/distance to work’, and ‘early retirement incentive/offer’. Respondents could report up to three reasons. The definition of exogenous job loss used here includes observations that reported being laid off due to a business closure. Workers who also stated that they quit or left for health reasons were excluded, but workers who stated they were also laid off or let go were included. For the control group, individuals who reported working for the same employer the entire time they were in the sample were used—i.e., individuals in the control group were not included if they ever quit their job or were laid off for any reason. Treated individuals are only in the analytic sample for two waves, or pre- and post-job loss. Control individuals can be in the analytic sample for multiple FIRS waves. In the whole observation period, there were 374 instances of involuntary job loss—i.e., the treatment group consisted of 374 workers who reported being laid off due to a business closure. The control group consisted of 11,558 observations on 3,564 workers.

A propensity score matched DiD model was used to evaluate the effect of job loss from a business closure

Difference-in-Differences (DiD) Approach

A DiD estimation combined with nonparametric kernel matching was used to estimate the average treatment effect on the treated (ATT) by genotype, or the change in BMI by genotype brought about by the job loss of those who actually lost their job. This approach compares individuals who have been laid off due to a business closure with a group of similar individuals who are still working for their same employer. To construct a control group with a similar distribution of covariates as the treatment group, the kernel-based matching estimator uses a distance-weighted average of all propensity scores in the control group to construct a counterfactual outcome for each individual in the treatment group. These weights were applied to the DiD regression model to obtain a balanced sample of treated and untreated individuals. The coefficients from the DiD regression were then used to estimate the ATT by mPGS and vPGS.

A traditional DiD setting assumes that after conditioning on a vector of observables X, the BMI of individuals in the treatment group would have evolved similarly over time to the BMI of individuals in the control group if they had never been laid off:

E[BMIᵢₜ−BMIᵢₜ₋₂|X,BC=1]|=E[BMI_{i′t}−BMI_{i′t-2}|X,BC=0]

Where BMIᵢₜ−BMIᵢₜ₋₂ refers to the change in BMI before and after the treatment, BC denotes the treatment group indicator (i.e., whether an individual lost their job due to a business closure), and i′ denotes an individual in the control group with the same characteristics as individual i in the treatment group. While conditioning on genotype and a rich set of covariates minimizes the possibility of violating this assumption, other systematic differences between the treated and control groups may remain even after conditioning on observables.

To minimize potential confounding from unobservable characteristics, the weights from propensity score matching (W) were used to reduce unmeasured differences between the treatment and control groups that could bias estimates:

E[BMIᵢₜ−BMIᵢₜ₋₂|W(X),BC=1]=E[BMI_{i′t}−BMI_{i′t-2}|W(X),BC=0]

Covariates used to estimate the propensity score, or the probability of treatment, were also included in the DiD regression model. Thus, coefficients from the regression-adjusted semiparametric DiD matching estimator are considered “doubly robust” because the estimator is consistent if the regression model or the propensity score model is correctly specified. As a result, the DiD matching estimator accounts for selection on observable and unobservable variables with time invariant effects, or the model allows for systematic differences between treatment and control groups even after conditioning on observables.

Difference-in-Differences (DiD) Empirical Strategy

The empirical strategy here can be broken down into three parts. First, propensity scores were estimated using a probit regression that regresses business closures on the mPGS and vPGS, as well as a rich set of covariates that are both standard in the job loss literature and satisfy the conditional independence assumption—i.e. they influence job loss and/or changes in BMI. In addition, it was only conditioned on observables that were unaffected by job loss (or the anticipation of it), or variables that were either fixed over time or measured in t−2. To avoid losing observations with missing information on a covariate, missing values were set equal to zero and included an additional dichotomous variable that is equal to one if the observation is missing. As a result, matching is not only on observed values but also on the missing data pattern. Throughout, the analysis was restricted to the region of common support, or the subset of individuals in the control group that were comparable to individuals in the treatment group. Specifically, treatment observations were dropped whose propensity score was greater than the maximum or less than the minimum propensity score of the controls.

The estimates from the probit regression were used to compute the weights for the control group with kernel matching, a nonparametric matching estimator that uses the weighted averages of all observations on common support to construct the counterfactual outcome. Specifically, the weight given to a non-treated individual j was in proportion to the closeness of their observables to treated individual i:

$$w ( i , j ) = \frac{K [ {P}_{j} - {P}_{i} ) / b ]}{{\sum }_{m ∈ BC = 0} K [ ( {P}_{m} - {P}_{i} ) / b ]}$$

Where P is the propensity score for individual i or j in the treated or control group, respectively, K[⋅] is the kernel function, and b is the bandwidth parameter. The program psmatch2 in Stata was used to compute w(i, j) with the Epanechnikov kernel function and a bandwidth of 0.06. In addition, when computing the weights, exact matching was performed on survey year and sex in t−2. This ensured 1) individuals who were laid off were matched with controls from the same time period, and 2) treated individuals were grouped with same-sex non-treated individuals.

In the final step, the weights from propensity score matching were incorporated into the DiD regression model:

BMIᵢₜ=α+λBCᵢₜ₋₂+ϕmPGSᵢ+ϑBCᵢₜ₋₂×mPGSᵢ+θvPGSᵢ+δBCᵢₜ₋₁×vPGSᵢ+τBMIᵢₜ₋₂+Xᵢₜ₋₂′β+εᵢₜ

Where BC is an indicator for job loss due to a business closing in the years between FIRS survey waves, or between t−2 and t for individual i, X is a vector of observable time invariant and variant covariates measured at t−2, including the first 10 principal components of the genetic data. BMIᵢₜ₋₂ was also included to control for baseline BMI, or to estimate deviations in BMI between t−2 and t. All regressions were estimated with robust standard errors.

Estimating Average Treatment Effects by Genotype

Estimated parameters from the DiD regression model were used to estimate the conditional mean or predicted BMI for treated and untreated individuals at various values of the mPGS and vPGS (FIG. 1). For example, the BMI for treated (BC=1) and untreated (BC=0) individuals with hypothetical mPGS and vPGS values at 0 and 1, respectively, would be estimated as follows:

E[BMIₜ|W(X),BC=1,vPGS=1,mPGS=1]=λ+ϕ+ϑ+θ+δ+τ+Xₜ₋₂β

E[BMIₜ|W(X),BC=0,vPGS=1,mPGS=1]=ϕ+θ+τ+Xₜ₋₂β

E[BMIₜ|W(X),BC=1,vPGS=0,mPGS=0]=λ+τ+Xₜ₋₂β

E[BMIₜ|W(X),BC=0,vPGS=0,mPGS=0]=τ+Xₜ₋₂β

From here, the ATT can be estimated by taking the difference in E[BMIₜ|W(X)] between treated and non-treated individuals with the same mPGS and vPGS values:

ATT_{vPGS=1,mPGS=1}=E[BMIₜ|W(X),BC=1]−E[BMIₜ|W(X),BC=0]λ+ϑ+δ

ATT_{vPGS=0,mPGS=0}=E[BMIₜ|W(X),BC=1]−E[BMIₜ|W(X),BC=0]=λ

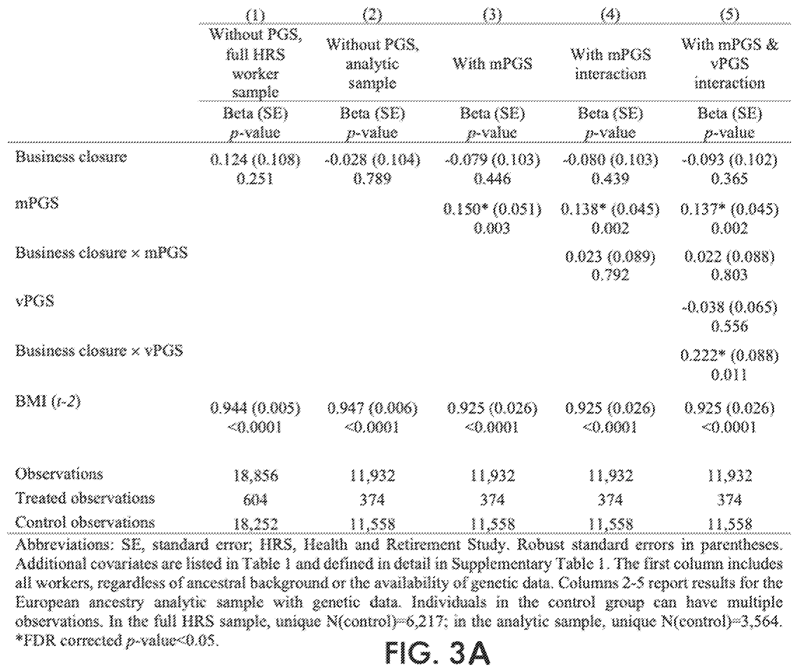

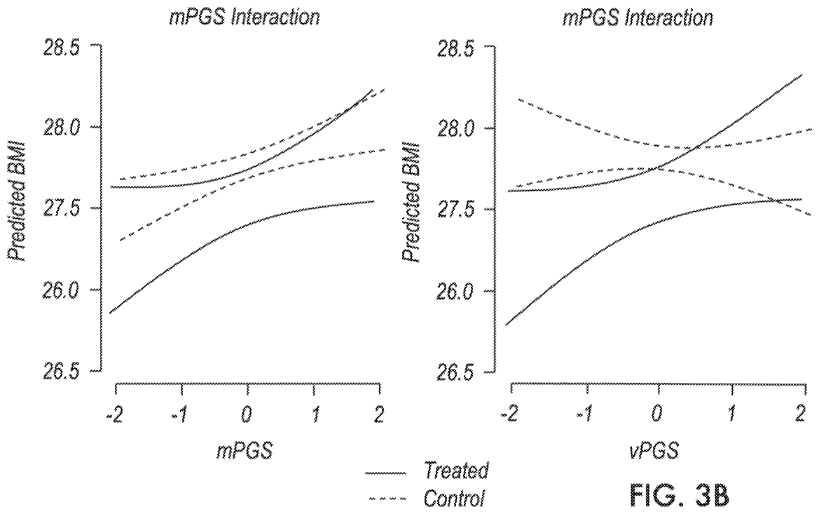

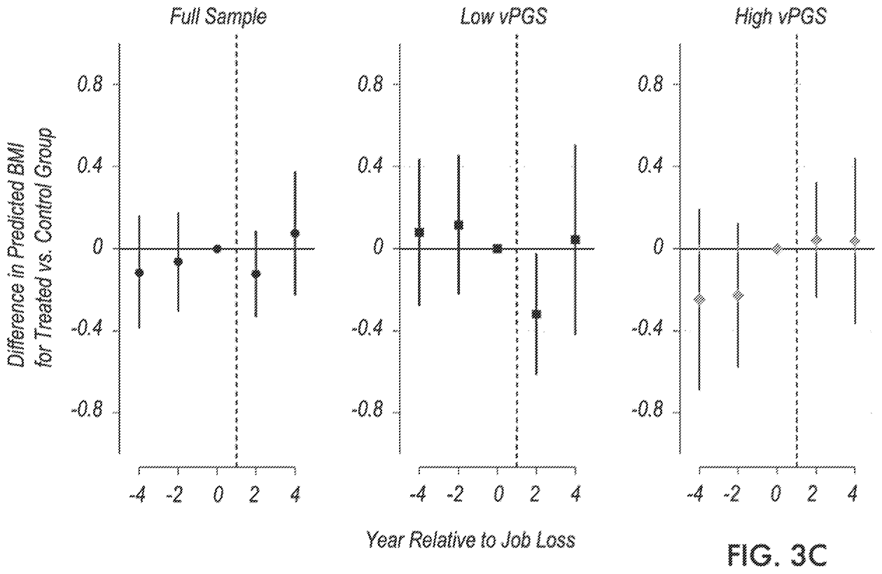

FIG. 3A shows separate propensity score adjusted DiD results from specifications with and without the mPGS and vPGS interactions. Columns 1-2 model changes in BMI without the mPGS and vPGS in the full HRS sample of workers aged 50-70 (1) and in the same-aged European ancestry analytic sample of workers with genotype data (2). Columns 3-4 add the main effect (3) and mPGS interaction effect (4), and Column 5 displays results from the full model with the mPGS and vPGS main effects and interaction effects. All specifications adjust for the conditioning variables used in the propensity score matching that are reported in Table 1. Results without PGSs in Columns 1 and 2 do not condition on genotype in the matching procedure or in the regression analysis. Coefficient p-values that pass FDR correction are denoted with an asterisk.

The results in Column 1 find a positive, but insignificant main effect from business closures in the full HRS sample on changes in BMI in all ancestry groups. Among genotyped, European ancestry respondents between the ages of 50 and 70 who report being in the labor force, the main effect is still insignificant but negative (Column 2). Columns 3-5 show that the inclusion of both the mPGS and vPGS is necessary to uncover a genetic main effect and an interaction effect: the mPGS captures a significant main effect of genotype on BMI (p=0.002), while the vPGS captures a significant G×E effect (p=0.011).

Graphically, this can be seen in FIG. 3B, which used estimated parameters from the DiD regression model to predict BMI at different values of the mPGS and vPGS for treated individuals in the wave following a job loss and for corresponding control individuals that were matched to treated individuals in the same HRS wave. In the mPGS figure, which plots predicted BMI based on the mPGS interaction model in Column 3, one can see differences in the predicted level of BMI by mPGS, but no significant differences between groups. Conversely, in the vPGS figure, which plots predicted BMI based on the vPGS interaction model in Column 5, there are no differences in the predicted level of BMI by vPGS, but as indicated by the cross-over shape of the interaction, there is suggestive evidence of a G×E interaction, or evidence of environmental moderation by vPGS for the treatment group relative to the control group in the post-treatment wave. Significant differences between treatment and control groups can only be seen in the lower half of the vPGS distribution; individuals below the mean appear more likely to lose weight as a result of a business closure relative to workers with similar vPGS scores. These results seem to indicate that less plastic individuals may adapt more slowly to environmental shifts, at least of the type studied herein.

An event time study (ETS) was conducted to assess the validity of the findings and to show the evolution in BMI by vPGS for the treatment and control groups up to four years post job loss. The assumption underlying the DiD research design is that in the absence of an involuntary job loss, BMI would have evolved similarly for the treatment and control groups (i.e., the “parallel trends” assumption).

Event Time Study Analysis

An event time study (ETS) model was estimated for individuals in the top and bottom 50% of the vPGS distribution using the following specification:

BMIᵢₜ=α+BCᵢ×Σ_{y=-4}_{_{y≠2}}²I(t−tᵢ*=y)+ϕmPGSᵢ+τBMIₜ₋₂+Xₜ₋₂′β+εᵢₜ.

This model is similar to the DiD model outlined above except the business closure term is replaced by a series of event terms that are the product of indicators for each HRS survey year (y) relative to the survey year the respondent reported a job loss tᵢ*, I(t−tᵢ*=y), and their treatment status (BO The omitted category is the survey year prior to treatment (y≠2). ETS results are also presented for the full sample that includes controls for the vPGS. Each estimate of λ_{y} gives the difference in BMI for treated individuals compared to non-treated individuals relative to this excluded year. If outcomes were evolving similarly for treated and untreated individuals prior to a business closure, the coefficient estimates for y<0 should be close to zero and not statistically significant.

FIG. 3C plots the coefficient estimates from the ETS model, which can be interpreted as the difference in BMI between treatment and control groups. The left panel of FIG. 3C indicates the presence of parallel trends in BMI prior to a business closure for the full sample—i.e., the difference between treatment and control groups is close to zero and not statistically significant. Separate event time study regressions were then estimated for respondents in the top 50% (right panel) and bottom 50% (middle panel) of the vPGS distribution to compare trajectories in BMI for treatment and control groups by vPGS. Similar to the results in FIG. 3B, suggestive evidence was found that individuals in the bottom 50% of the vPGS distribution have a lower BMI on average compared to the control group (p=0.035). Effects did not appear to persist in the next HRS interview wave at t+2, or up to four years post job loss.

In this example, results indicate that an unexpected job loss did not seem to affect differences in BMI by mPGS; individuals with higher mPGS had higher levels of BMI regardless of whether they were in the treatment or control group. However, one can see suggestive evidence of genetic moderation by vPGS such that individuals in the treatment group varied in their genetic propensity for weight gain or loss, whereas similarly matched individuals in the control group did not. Genetic moderation is particularly pronounced in the lower half of the vPGS distribution; less plastic individuals in the bottom 50% appear to adjust more slowly to environmental changes, resulting in minor weight loss compared to similarly matched individuals in the control group. Results from the event time study analysis show that changes in BMI were detectable up to two years post job loss, but did not persist in the subsequent HRS wave, or up to four years post job loss.

### Example 4 (Comparing mPGS Versus vPGS as Moderators of a UK Education Reform)

The two preferred plasticity scores from Example 1 (one summarizing “dispersion” effects; the other constructed from sibling variation) were used to study heterogeneous effects of a large-scale education reform initiated in 1972 in England, Scotland, and Wales that extended how long students were legally required to stay in school from 15 to 16 years old. Using Barcellos' regression discontinuity design (see Barcellos, S. H., et al., “Education can reduce health differences related to genetic risk of obesity”, Proceedings of the National Academy of Sciences, 115(42):E9765— E9772 (2018), the entirety of which is incorporated by reference herein), the extent to which the two effective vPGS are able to detect different forms of genetic moderation of this educational shock than the standard levels polygenic scores can be evaluated. Two different cases using the same reform as the exogenous environmental context were examined: one where, following the Barcellos, the moderating role that the genetic risk of obesity plays in the relationship between education on body size was examined, and another where the influence of genetic plasticity on downstream educational outcomes was evaluated. Both outcomes are affected by gene and environment interactions but differ in the kinds of G×E effects they exhibit: while the effect for body size is the result of outcome moderation, the latter results from plasticity.

More specifically, following Barcellos, 2SLS was used to first instrument whether someone stayed in school until 16 years of age (Educ16) with whether they were younger than 16 when the reform went into place (post reform), and were therefore legally required to stay the extra year. This residualized version of Educ16 is then interacted in the second step with BMI PGS to evaluate whether there is an interaction between educational attainment and genes as they affect health outcomes. A reduced form regression is also reported, where PGS is directly interacted with the post reform variable.

The main health outcome is Body Size—a weighted combination of BMI, waist-to-hip ratio, and body fat percentage. In a separate NBER preprint, Barcellos et al, “Distributional effects of education on health”, Technical report, National Bureau of Economic Research (2019) (the entirety of which is incorporated by reference herein), Barcellos identified the point in the Body Size distribution at which they should have the most power to detect an effect. This same distributional threshold can be used to create an Above Threshold version of Body Size, the results for which can be compared to the continuous version of the outcome.

In a second set of analyses, the role of genes in the impact of an additional year of education on downstream educational outcomes are examined. Here, rather than instrumenting educational attainment, the effect of one's genotype on education outcomes differs depending on whether one was born before or after the reform is specifically considered. This is akin to the reduced form regressions reported for BMI. The previous literature found no effect of the reform on the likelihood of attending college, and this example focuses on the outcomes of those who left school at the age of 18 or younger. The effect of the reform on four educational outcomes, previously used in the literature, are examined. First, whether the respondent left school at age 16 or later is considered, since despite the reform some students still opted out of attending college through age 16 (Left School 16 or later). Second, whether respondents achieved any certifications as a result of their education (Certification) is considered. For the last two outcomes, whether they achieved specific certifications: O-levels or CSE (which were equivalent and later replaced by the GCSE in 1988) and A-levels, are considered.

Controls, for both sets of models, include a quadratic term for the number of days that passed from when the respondent was born until the time of reform (to factor out any time trends), dummy variables for the month born, sex, age at time of assessment in days, age squared, dummy variables for country of birth, the first 15 PCs, mPGS, the interaction between those PCs and Educ 16 (or, in the reduced form, Post Reform), and the interaction between mPGS with Educ16. Triangular kernel weights were used to assign more weight to observations closer to the reform and time trends were allowed to vary before and after the reform (Barcellos et al., 2018).

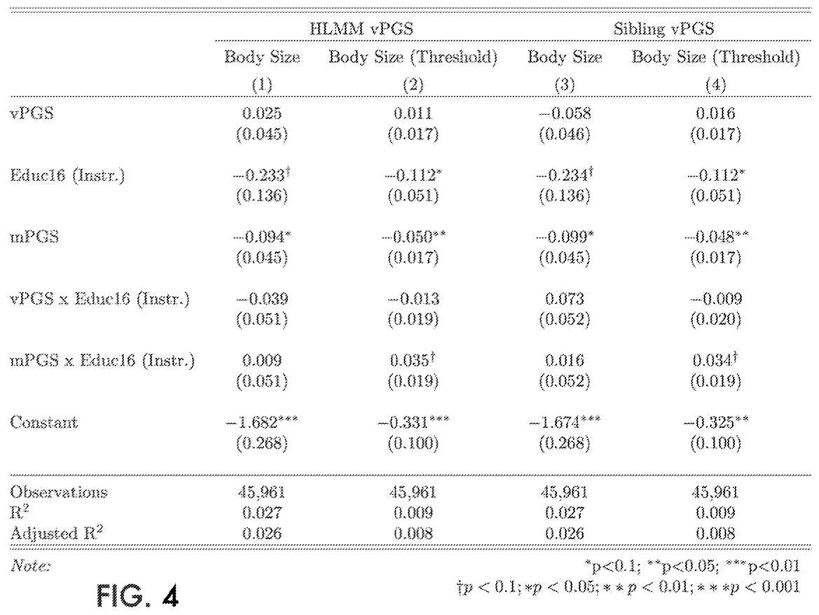

For the body size models, presented in FIG. 4, the interaction between mPGS and being exposed to the reform is the only statistically significant result. None of the polygenic scores for plasticity show a significant interaction, with the exception of Squared Z-Score vPGS. This result is likely due to the high correlation between the Squared Z-Score vPGS and mPGS.

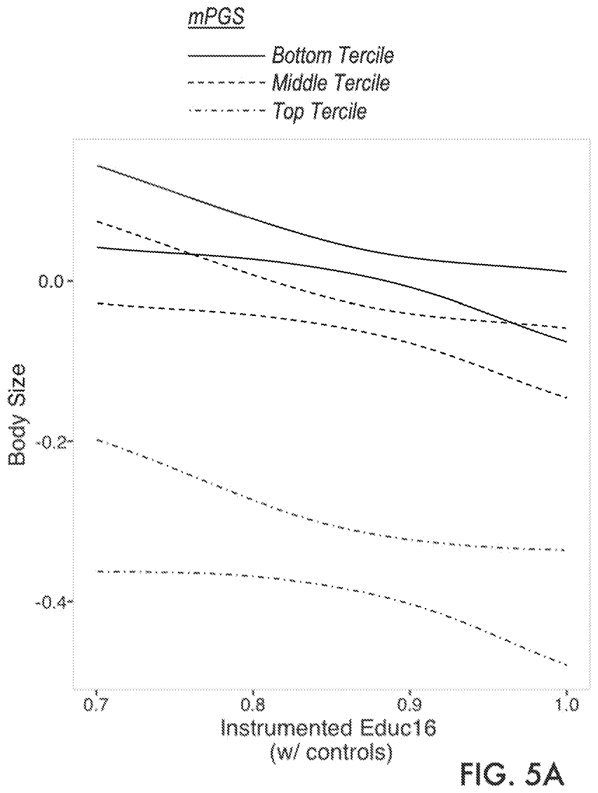

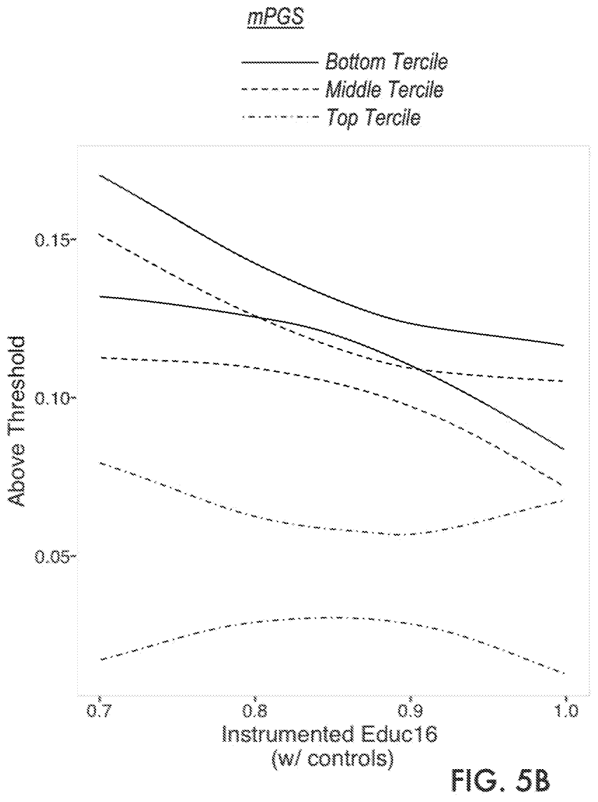

These results suggest that outcome moderation (rather than plasticity) is the main form that genetic moderation of the education reform takes when impacting these measures of health. Put differently, and as visualized in FIGS. 5A and 5B, the reform has larger impacts on reducing obesity-related measures among those with already-lower genetic propensities towards obesity.

Embodiments of the present disclosure are described in detail with reference to the figures wherein like reference numerals identify similar or identical elements. It is to be understood that the disclosed embodiments are merely examples of the disclosure, which may be embodied in various forms. Well known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

Those skilled in the art will recognize or be able to ascertain using no more than routine experimentation many equivalents to the specific embodiments of the invention described herein. Such equivalents are intended to be encompassed by the following claims.

What is claimed:

1. A method for calculating variation in a genetic score, comprising:
generating at least one variance polygenic score (vPGS) of phenotypic information by:
performing a regression to predict a squared Z-score for each outcome of the phenotypic information, and summing the weights of the regressions into a single index for scoring individuals' DNA variants into the variation polygenic score;
performing a regression to predict a statistical measure of spread within a familial relationship of the phenotypic information, and summing the weights of the regressions into a single index for scoring individuals' DNA variants into the variation polygenic score;
performing a mean-variance vQTL analysis for variance heterogeneity of the phenotypic information, and summing the weights of the regressions into a single index for scoring individuals' DNA variants into the variation polygenic score;
performing a mean-variance vQTL analysis for variance effects of phenotypic information, and summing the weights of the regressions into a single index for scoring individuals' DNA variants into the variation polygenic score; or
a combination thereof.

2. The method according to claim 1, further comprising selecting or defining the one or more outcomes, the one or more outcomes being present in the phenotypic information.

3. The method according to claim 2, wherein the one or more outcomes comprise height, body mass index, systolic blood pressure, diastolic blood pressure, number of alcoholic drinks consumed per a given time period, number of cigarettes consumed per a given time period, depression symptomology score, cognitive test score, educational attainment, and/or number of children born.

4. The method according to claim 1, wherein generating the at least one vPGS comprises calculating a statistical measure of spread within a familial relationship of phenotypic information, and wherein the statistical measure of spread comprises one of standard deviation, Levene's distance, and range.

5. The method according to claim 1, further comprising determining whether the at least one vPGS can capture genetic contributions to variability in an outcome, distinct from genetic contributions to levels of an outcome.

6. The method according to claim 5, further comprising applying the weights determined from performing the regression to a different data set.

7. The method according to claim 1, further comprising generating a mean polygenic score (mPGS) by running a regression using the phenotypic information to predict an inverse normal transformation for each outcome of one or more outcomes, such that the weights reflect the contribution of each genetic locus of a plurality of genetic loci to the mean level of the outcome, and summing the weights of the regression into the mPGS.

8. The method according to claim 7, further comprising comparing the mPGS and vPGS scores.

9. The method according to claim 1, further comprising selecting at least one participant for a trial based on the vPGS score.

10. A system, comprising:
a processor; and
a non-transitory computer readable storage medium containing instructions that, when executed, cause the processor to:
generate at least one variance polygenic score (vPGS) by:
performing a regression to predict a squared Z-score for each outcome of the phenotypic information, and summing the weights of the regressions into a single index for scoring individuals' DNA variants into the variation polygenic score;
a regression to predict a statistical measure of spread within a familial relationship of the phenotypic information, and summing the weights of the regressions into a single index for scoring individuals' DNA variants into the variation polygenic score;
a mean-variance vQTL analysis for variance heterogeneity of the phenotypic information, and summing the weights of the regressions into a single index for scoring individuals' DNA variants into the variation polygenic score;
a mean-variance vQTL analysis for variance effects of phenotypic information, and summing the weights of the regressions into a single index for scoring individuals' DNA variants into the variation polygenic score; or
a combination thereof.

11. The system according to claim 10, wherein the processor is further configured to receive information selecting or defining the one or more outcomes, the one or more outcomes being present in the phenotypic information.

12. The system according to claim 10, wherein generating the at least one vPGS comprises calculating a statistical measure of spread within a familial relationship of phenotypic information, and wherein the statistical measure of spread comprises one of standard deviation, Levene's distance, and range.

13. The system according to claim 10, wherein the processor is further configured to determine whether the at least one vPGS can capture genetic contributions to variability in an outcome, distinct from genetic contributions to levels of an outcome.

14. The system according to claim 10, wherein the processor is further configured to generate a mean polygenic score (mPGS) by running a regression using phenotypic information to predict an inverse normal transformation for each outcome of one or more outcomes, such that the weights reflect the contribution of each genetic locus of a plurality of genetic loci to the mean level of the outcome, and summing the weights of the regression into the mPGS.

15. The system according to claim 14, wherein the processor is further configured to compare the mPGS and the vPGS scores.
